(19) United States
(12) Patent Application Publication
(10) Pub. No.: US 2023/0066882 A1
(43) Pub. Date: Mar. 2, 2023

(54) Stable Reporter Flavivirus

(71) Applicant: Board of Regents, the University of Texas System, Austin, TX (US)

(72) Inventors: Coleman Baker, Galveston, TX (US); Xuping Xie, Galveston, TX (US); Pei-Yong Shi, Galveston, TX (US)

(21) Appl. No.: 17/412,900

(22) Filed: Aug. 26, 2021

(51) Int. Cl. C12N 7/00; C12N 9/02

(52) U.S. Cl. CPC C12N 7/00; C12N 9/0069; C12N 2770/24121

(57) ABSTRACT

Certain embodiments are directed to a stable recombinant flavivirus nucleic acid having a heterologous reporter cassette, the reporter cassette having a 5′ end, a nucleotide segment encoding a reporter, and a 3′ end; the 5′ end of the reporter cassette encoding 25 to 38 amino acids of a flavivirus capsid protein; the 3′ end of the reporter cassette encoding 25 to 38 amino acids of a flavivirus capsid protein.

### STATEMENT REGARDING FEDERALLY FUNDED RESEARCH

This invention was made with government support under U19AI142759 awarded by the National Institutes of Health (NIH) and U01CK0000512 awarded by the Center for Disease Control (CDC). The government has certain rights in the invention.

### REFERENCE TO SEQUENCE LISTING

A sequence listing required by 37 CFR 1.821-1.825 is being submitted electronically with this application. The sequence listing is incorporated herein by reference.

### BACKGROUND

Viruses from the arthropod-borne genus Flavivirus afflict people across the globe causing febrile, neurologic, and hemorrhagic disease¹. Notable among the flaviviruses are the four serotypes of dengue virus (DENV), which cause an estimated 96 million symptomatic infections yearly², Japanese encephalitis virus (JEV) which causes the annual loss of 709,000 disability-adjusted life years³, the recently emerged Zika virus (ZIKV), which has become associated with congenital malformations⁴, and yellow fever virus (YFV), which periodically emerges from its sylvatic transmission cycle to start an urban transmission cycle⁵. Concerted efforts by scientists and clinicians have brought about vaccines for YFV, JEV, and DENV^{6,7} though effective antiviral drugs have yet to be approved. Reporter flaviviruses, first published in 2003⁸, have been critical for high-throughput antiviral compound screens^{9,10} host and virus pathogenesis studies ¹¹, and serological diagnosis ^{12,13} . Despite these advances, reporter flaviviruses suffer from genetic instability during longer periods of growth or passaging, thought to be primarily mediated by recombination^{14,15}.

Reporter genes are routinely engineered at the beginning of the single open reading frame of the viral polyprotein, between the 5′ UTR and the capsid gene, as first described using YFV ^{16.} RNA signals that aid in genome cyclization, which is essential for viral replication, and facilitate translation are continuous from the 5′ UTR into the beginning of the capsid. These signals must function together, therefore it is necessary to duplicate a portion of the capsid gene and place it upstream of the inserted reporter gene. Until recently, efforts to stabilize these constructs centered on reducing homology between the duplicated capsid sequences by codon scrambling in an effort to reduce homologous recombination^{16,17}. This also had the benefit of expunging the cis-acting elements in the full capsid sequence, leaving only the upstream elements. Two additional methods for stabilizing reporter flaviviruses have been newly developed, both focusing on blocking recombined reporter viruses from continued infection. Volkova and colleagues report a single-nucleotide insertion to the duplicated capsid portion of a reporter ZIKV that minimally perturbs critical RNA elements but causes a +1 frameshift¹⁸. If recombination occurs between the duplicated capsid sequences that flank the reporter gene, this frameshift mutation is incorporated into the viral polyprotein and causes mistranslation, effectively taking out recombined viruses from the population. We developed a related method for reporter ZIKV and YFV using recombination-dependent lethal mutations in the duplicated capsid¹⁹. These lethal mutations stop viral particle formation if recombination brings them into the viral polyprotein.

The length of capsid duplications in different flavivirus reporter constructs that have been reported varies from 25, to 33 or 34, 38, 50, or even the full capsid^{10,16-18,20-24} Shorter lengths are tolerated by some viruses and not by others. At the onset of this investigation, there was no published comparison of the effect of capsid duplication length and its effect on stability. It was believed that shorter capsid repeats were preferred because the shorter homologous sequence minimizes homologous recombination. It is hypothesized that an optimal length of capsid duplication is required for efficient viral replication; a shortened capsid duplication imposes a selection pressure on viral replication, leading to undesired recombination and deletion of the engineered reporter gene. The goal of this study is to test this hypothesis by engineering different lengths of capsid duplication and investigating the length effect on the stability of the reporter gene in various flaviviruses. Indeed, an optimal length of capsid duplication of 34 or 38 amino acids were identified that can increase the reporter gene stability for at least ten rounds of cell culture passages. Taking this new approach, the inventors have developed a panel of long-term stable NanoLuc-tagged flaviviruses, including the four serotypes of DENY, JEV, YFV, and ZIKV. In addition, it is demonstrated the reporter flaviviruses can be used for rapid antibody neutralization testing and antiviral drug discovery. Taken together, the results have established a previously unrecognized approach to generate stable reporter flaviviruses that are useful for research and countermeasure development.

### SUMMARY

Mosquito-transmitted flaviviruses cause widespread disease across the world and medical countermeasures have yet to be proven effective. To provide better molecular tools for drug screens and pathogenesis studies, a new approach to produce stable reporter-tagged flaviviruses, including dengue virus serotypes 1-4, Japanese encephalitis virus, yellow fever virus, West Nile virus, and Zika virus is described herein. Since the reporter gene is often engineered at the flaviviral capsid gene region, the capsid sequence must be duplicated to flank the reporter gene; such capsid duplication is essential for viral replication. The conventional approach for stabilizing reporter flaviviruses has been to shorten or modify the duplicated capsid sequence to minimize homologous recombination. No study has examined the effects of capsid duplication length on reporter virus stability. Described herein is the optimal length of capsid duplication (34 or 38 amino acids without any other modifications) that can stabilize reporter flaviviruses. NanoLuc-tagged flaviviruses were stable after ten rounds of cell culture passaging. Mechanistically, the optimal length of capsid duplication may contain all the cis-acting RNA elements required for viral RNA replication, thus reducing the selection pressure for recombination. Certain embodiments are directed to improved methods of constructing optimal reporter flaviviruses and the resultant reporter flaviviruses.

Certain embodiments are directed to a recombinant flavivirus encoding a polyprotein having an amino terminal reporter element comprising, amino to carboxy, a first C25 to C38 capsid element, a reporter, a 2A self-cleaving element (e.g., thosea asigna virus 2A, T2A), and a second C25 to C38 capsid element.

Certain embodiments are directed to a recombinant flavivirus comprising, consisting essentially of or consisting of a nucleic acid sequence that is at least 90, 95, 96, 97, 98, 99, to 100% identical to SEQ ID NO:13, 15, 17, 19, 21, 23, 25, 27, 29, or 31, and encoding a polyprotein having the amino acid sequence that is at least 90, 95, 96, 97, 98, 99, to 100% identical to SEQ ID NO:14, 16, 18, 20, 22, 24, 26, 28, 30, or 32, respectively.

SEQ ID NO:13 includes a reporter cassette including from 5′ to 3′ a first C25 element (nucleotides 107-181 encoding amino acids 1-25 of SEQ ID NO:14); a representative reporter (nanoLuc report nucleotides 181-694 encoding amino acids 26-196 of SEQ ID NO:14); a T2A element (nucleotides 695-757 encoding amino acids 197-217 of SEQ ID NO:14); and a second C25 element (nucleotides 758-832 encoding amino acids 218-242 of SEQ ID NO:14). The reporter cassette is in frame with the polyprotein.

SEQ ID NO:15 includes a reporter cassette including from 5′ to 3′ a first C38 element (nucleotides 107-220 encoding amino acids 1-38 of SEQ ID NO:16); a representative reporter (nanoLuc reporter nucleotides 221-733 encoding amino acids 39-209 of SEQ ID NO:16); a T2A element 734-796 encoding amino acids 210-230 of SEQ ID NO:16); and a second C38 element (nucleotides 797-910 encoding amino acids 231-268 of SEQ ID NO:16). The reporter cassette is in frame with the polyprotein.

SEQ ID NO:17 includes a reporter cassette including from 5′ to 3′ a first C38 element (nucleotides 95-208 encoding amino acids 1-38 of SEQ ID NO:18); a representative reporter (nanoLuc reporter nucleotides 209-721 encoding amino acids 39-209 of SEQ ID NO:18); a T2A element 722-784 encoding amino acids 210-230 of SEQ ID NO:18); and a second C38 element (nucleotides 785-898 encoding amino acids 231-268 of SEQ ID NO:18). The reporter cassette is in frame with the polyprotein.

SEQ ID NO:19 includes a reporter cassette including from 5′ to 3′ a first C38 element (nucleotides 97-210 encoding amino acids 1-38 of SEQ ID NO:20); a representative reporter (nanoLuc reporter nucleotides 211-723 encoding amino acids 39-209 of SEQ ID NO:20); a T2A element 724-786 encoding amino acids 210-230 of SEQ ID NO:20); and a second C38 element (nucleotides 787-900 encoding amino acids 231-268 of SEQ ID NO:20). The reporter cassette is in frame with the polyprotein.

SEQ ID NO:21 includes a reporter cassette including from 5′ to 3′ a first C38 element (nucleotides 95-208 encoding amino acids 1-38 of SEQ ID NO:22); a representative reporter (nanoLuc reporter nucleotides 209-721 encoding amino acids 39-209 of SEQ ID NO:22); a T2A element 722-784 encoding amino acids 210-230 of SEQ ID NO:22); and a second C38 element (nucleotides 785-898 encoding amino acids 231-268 of SEQ ID NO:22). The reporter cassette is in frame with the polyprotein.

SEQ ID NO:23 includes a reporter cassette including from 5′ to 3′ a first C38 element (nucleotides 103-216 encoding amino acids 1-38 of SEQ ID NO:24); a representative reporter (nanoLuc reporter nucleotides 217-729 encoding amino acids 39-209 of SEQ ID NO:24); a T2A element 730-792 encoding amino acids 210-230 of SEQ ID NO:24); and a second C38 element (nucleotides 793-906 encoding amino acids 231-268 of SEQ ID NO:24). The reporter cassette is in frame with the polyprotein.

SEQ ID NO:25 includes a reporter cassette including from 5′ to 3′ a first C25 element (nucleotides 119-193 encoding amino acids 1-25 of SEQ ID NO:26); a representative reporter (nanoLuc reporter nucleotides 194-706 encoding amino acids 26-196 of SEQ ID NO:26); a T2A element 707-769 encoding amino acids 197-217 of SEQ ID NO:26); and a second C25 element (nucleotides 770-844 encoding amino acids 218-242 of SEQ ID NO:26). The reporter cassette is in frame with the polyprotein.

SEQ ID NO:27 includes a reporter cassette including from 5′ to 3′ a first C38 element (nucleotides 119-232 encoding amino acids 1-38 of SEQ ID NO:28); a representative reporter (nanoLuc reporter nucleotides 233-745 encoding amino acids 39-209 of SEQ ID NO:28); a T2A element 746-808 encoding amino acids 210-230 of SEQ ID NO:28); and a second C38 element (nucleotides 809-922 encoding amino acids 231-268 of SEQ ID NO:28). The reporter cassette is in frame with the polyprotein.

SEQ ID NO:29 includes a reporter cassette including from 5′ to 3′ a first C34 element (nucleotides 96-197 encoding amino acids 1-34 of SEQ ID NO:30); a representative reporter (nanoLuc reporter nucleotides 198-710 encoding amino acids 35-205 of SEQ ID NO:30); a T2A element 711-773 encoding amino acids 206-226 of SEQ ID NO:30); and a second C34 element (nucleotides 774-875 encoding amino acids 227-260 of SEQ ID NO:30). The reporter cassette is in frame with the polyprotein.

SEQ ID NO:31 includes a reporter cassette including from 5′ to 3′ a first C33 element (nucleotides 97-195 encoding amino acids 1-33 of SEQ ID NO:32); a representative reporter (nanoLuc reporter nucleotides 196-708 encoding amino acids 34-204 of SEQ ID NO:32); a T2A element 709-771 encoding amino acids 205-225 of SEQ ID NO:32); and a second C33 element (nucleotides 772-870 encoding amino acids 226-258 of SEQ ID NO:32). The reporter cassette is in frame with the polyprotein.

Certain embodiments are directed to a recombinant flavivirus comprising a heterologous reporter cassette, the heterologous reporter cassette having a 5′ end, a nucleotide segment encoding a reporter, and a 3′ end; the 5′ end of the reporter cassette encoding 25 to 38 amino acids of an amino terminus of flavivirus capsid protein; the 3′ end of the reporter cassette encoding 25 to 38 amino acids of an amino terminus of the flavivirus capsid protein, wherein the recombinant flavivirus is stable. In certain aspects, the recombinant flavivirus further comprises a nucleic acid segment encoding a 2A self-cleaving sequence position immediately 3′ of the reporter and immediately 5′ of the nucleotide segment encoding the second 25 to 38 amino acids of a flavivirus capsid protein. The 2A self-cleaving segment can be a T2A segment. In certain aspects, the flavivirus is a Dengue virus, Zika virus, or Japanese encephalitis virus. The reporter can be, but is not limited to a NanoLuc, EGFP, mCherry, mScarlet, mNeonGreen, or Renilla Luciferase reporter. In other aspects, the recombinant flavivirus has a nucleic sequence that is at least 98% to 100% identical to SEQ ID NO:13, 15, 17, 19, 21, 23, 25, 27, 29, or 31. In certain aspects the recombinant flavivirus encodes a polyprotein that is at least 95% to 100% identical to the amino acid sequence of SEQ ID NO:14, 16, 18, 20, 22, 24, 26, 28, 30, or 32.

Certain embodiments are directed to a recombinant flavivirus polyprotein comprising an amino terminal segment comprising a heterologous reporter, the reporter being flanked by an amino terminal first capsid segment corresponding to capsid segment comprising 25 to 38 amino terminal amino acids of the flavivirus capsid protein and a carboxy terminal second capsid segment corresponding to a capsid segment comprising 25 to 38 amino of the amino terminus of the flavivirus capsid protein. The polyprotein can further comprise a 2A self-cleaving segment between the reporter and the second capsid segment.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of and “consisting of exclude any element, step, or component not specified. For example, “consists of or “consisting of used in a claim would limit the claim to the components, materials or steps specifically recited in the claim except for impurities ordinarily associated therewith (i.e., impurities within a given component). When the phrase “consists of or “consisting of appears in a clause of the body of a claim, rather than immediately following the preamble, the phrase “consists of or “consisting of limits only the elements (or components or steps) set forth in that clause; other elements (or components) are not excluded from the claim as a whole.

As used herein, the transitional phrases “consists essentially of and “consisting essentially of are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of occupies a middle ground between “comprising” and “consisting of′.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-D. Panel of Reporter Flaviviruses. FIG. 1A. Scheme of NanoLuc insertion in the flavivirus genome. The duplicated capsid portion varies from 25 to 38 amino acids, depending on the virus. The white beginning of the capsid gene indicates the codon scrambling to reduce homology with the duplicated portion. FIG. 1B. Virus, corresponding number of capsid amino acids duplicated, and representative IFA images from Day 1 to 3 post electroporation. The pan-flavivirus envelope antibody 4G2 was used to probe for virus and cells were counterstained with DAPI. FIG. 1C. Focus-forming assay after 4-day infection on Vero cells. FIG. 1D. Genetic stability results after two independent series of ten passages in Vero cells. Viral RNAs from each passage were used as templates in an RT-PCR reaction with primers that spanned the 5′ UTR to the capsid. Full-length band sizes are as follows ZIKV: 1,162 bp; DENV1: 1,086 bp; DENV2: 1,128 bp; DENV3: 1,126 bp; DENV4: 1,131 bp; YFV: 1,282 bp; JEV: 1,360 bp.

FIGS. 2A-C. Extended Capsid Duplication. FIG. 2A. Virus, corresponding number of duplicated capsid amino acids, and representative Day 1 to Day 3 IFA images. The anti-flavivirus envelope antibody 4G2 was used. FIG. 2B. Focus-forming assay on Vero cells after four-day infection. FIG. 2C. Reporter gene retention after ten passages on Vero cells. RT-PCR products covering the reporter gene are shown for the two independent passaging series. ZIKV C50 passaging was discontinued after P2 due to loss of reporter gene. Band sizes corresponding to the full-length reporter gene are as follows ZIKV C38: 1,201 bp; ZIKV C50: 1,237 bp; YFV C38: 1,321 bp.

FIGS. 3A-C. Effect of Capsid Duplication Length on Viral Growth. Multi-step growth kinetics (MOI 0.01, n=3) on Vero cells, using focus-forming assay to quantify growth for FIG. 3A. ZIKV C25, C38, and C50; FIG. 3B. YFV C25 and C38; C. DENV1-4 and JEV. 2-way repeated measures ANOVA with Tukey's multiple comparisons test was used to assess significance for FIG. 3A and FIG. 3C. 2-way repeated measures ANOVA with Sidak's multiple comparisons test was used for FIG. 3B (p>0.5=ns, p<0.5=*, p<0.1=**, p<0.01=***, p<0.001=****).

FIGS. 4A-4C Neutralization and Antiviral Assays. FIG. 4A. NT5o values of a panel of mouse sera tested with the stable reporter viruses. For ZIKV and YFV NT50 tests, the C38 virus was used in both instances. FIGS. 4D-E. EC5o results from testing NITD008 against the panel of stable reporter viruses in both graphical and table formats, respectively.

FIGS. 5A-B. ZIKV C38 Nano in Mosquitoes. FIG. 5A. Aedes aegypti mosquitoes were micro-injected with 50 FFU ZIKV C38 Nano (n=30 per day). On days 8, 12, and 18, whole mosquitoes were collected and individually homogenized in PBS. Samples were analyzed by both qPCR (left panel) or luciferase assay (right panel). FIG. 5B. Aedes aegypti mosquitoes (n=50 per group) were inoculated by membrane blood-feeding on sheep's blood spiked with 2×10⁶ FFU/mL ZIKV C38 Nano. Mosquitoes were then analyzed as in panel FIG. 5A. For the luciferase assay, clean (uninfected) mosquitoes were used as a control. Values for luciferase activity are reported relative to background (no mosquito) levels. ANOVA with Tukey's post-hoc test was used to assess significant differences in all panels (p>0.5=ns, p<0.5=*, p<0.1=**, p<0.01=***, p<0.001=****).

FIGS. 6A-B. In vitro ligation scheme for DENV3 (FIG. 6A) and DENV4 (FIG. 6B).

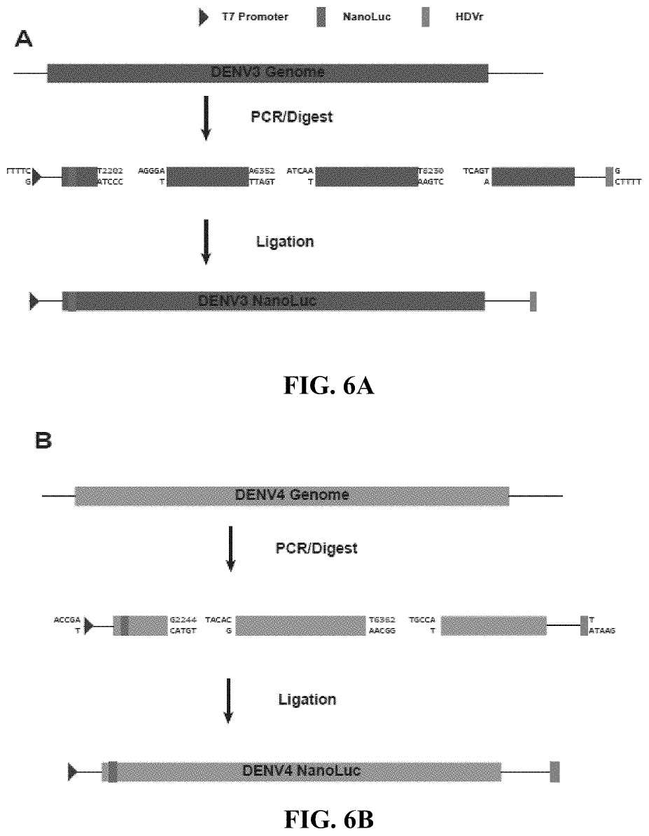

The nucleotide positions of each fragment junction are indicated in red according to GenBank accession numbers EU482459 and FN429920, respectively.

FIGS. 7A-B. FIG. 7A. Capsid duplication length of WNV along with IFA images stained with 4G2 and DAPI. FIG. 7B. Stability results of passaged WNV-Nano, full length size 1,140 bp

FIGS. 8A-B. FIG. 8A. Scheme for passaging reporter viruses. PO is used to denote viral stock recovered after electroporation. 500₁1.1_, of PO was used to inoculate a T75 flask of confluent Vero cells. Infection was allowed to proceed until cell death was observed. This was repeated until P10 virus was obtained. Viral RNA was obtained from each passage and used in an RT-PCR reaction that bridged the reporter gene. These products were run on an agarose gel to observe band size. FIG. 8B. Focus-forming assay using WT ZIKV on Vero cells after a 4-day infection.

FIG. 9. Growth kinetics comparison of ZIKV C38 and WT ZIKV on Vero cells (MOI 0.01, n=3). Significant differences were assessed with 2-way repeated measures ANOVA with Sidak's multiple comparisons test (p>0.5=ns, p<0.5=*, p<0.1=**, p<0.01=***, p<0.001=****). Data for ZIKV C38 is the same as shown in FIG. 3A.

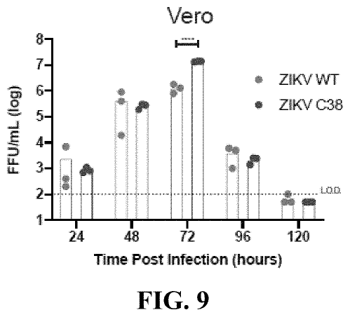

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Flavivirus reporter constructs have been notoriously unstable since they were first reported¹⁴′³³. Improvements in design¹⁶ have increased the stability, but previous efforts have fallen short of the high standard of ten passages. Here is described a panel of reporter flaviviruses, e.g., NanoLuc-tagged flaviviruses, with stability to at least ten passages in cell culture, which is double the passages routinely reported. It was found that C38 ZIKV and YFV reporter viruses were more stable than their C25 counterparts, and in the case of ZIKV, C38 had a distinct replication advantage. Previous hypotheses for constructing reporter flaviviruses assumes that shorter capsid duplication lengths would be more stable, due to a shorter stretch of homologous sequence. These results challenge that assumption, suggesting that C38 is optimal. The establishment of a stable reporter virus system will greatly facilitate the production of reporter virus in cell culture through viral infection and amplification rather than transfection of viral RNA transcribed from its infectious cDNA plasmid. The ease of stable reporter virus production enables potential high-throughput flavivirus neutralization testing and antiviral screening, as recently demonstrated for reporter SARS-CoV-2³⁴′³⁵.

Many different cis-acting elements present in the flavivirus capsid coding region have been mapped, including the 5′ cyclization sequence (CS)³⁶, the capsid hairpin (cHP)³⁷, the 5′ downstream of AUG region (DAR)³⁸, and the downstream of 5′ cyclization sequence pseudoknot (DCS-PK)³⁹. These elements work together to regulate RNA translation, genome cyclization, and viral replication. C25 includes all of those elements except the full DCS-PK, a pseudoknot that has been modeled in various flavivirus genomes, including ZIKV⁴⁰, and experimentally been found to aid viral replication in DENV2⁴¹ and DENV4³⁹. Extending C25 to C38 includes the full DCS-PK, which may explain the increased replication capacity of ZIKV C38 compared to ZIKV C25. We hypothesize that the lack of the DCS-PK in ZIKV C25 caused increased selective pressure and helped drive recombination. Inclusion of DCS-PK in ZIKV C38 lessened this selective pressure, expanding its stability. In contrast, YFV C38 replicated very similarly to YFV C25, supporting a model that YFV lacks, or has a shortened, DCS-PK³⁹. Despite this, the lengthened capsid duplication still had a positive effect on YFV stability.

Previous work with reporter ZIKV identified C50 as an optimal length for capsid duplication in relation to replication but its effect on stability was not independently tested¹⁸. C38 performed remarkably better in both stability and viral replication when compared to C50. The discrepancy could be due to different ZIKV strains, different sequences flanking the reporter gene, and the absence of a frameshift mutation which was included to help block recombination. ZIKV C50 includes the DCS-PK, along with the other required replication elements and as such would be expected to replicate similar to ZIKV C38. The extra capsid amino acids C39-050, which contain residues shown to be important in capsid dimerization⁴², could allow the C50 capsid fragment to interfere with full-length capsid, thus explaining the attenuation of C50 compared to C38. This selective pressure, along with a larger region for possible recombination, could also be driving the poor stability seen during passaging.

The inventors used sera from known virus-infected mice to demonstrate the utility of the reporter virus for neutralization testing. The reporter virus neutralization assay has been optimized in a 96-well format for high-throughput testing. For clinical use of the reporter neutralization testing, the assay can be validated using patient sera with well-defined viral infections. The validation can be achieved by comparing the antibody neutralizing titers derived from the conventional plaque reduction neutralization test (PRNT) with those derived from the reporter virus assay.

Together, these results demonstrate that extending the portion of capsid duplicated to make ZIKV and YFV reporter viruses can increase their stability and in the case of ZIKV enhance its replication capabilities in mammalian cells and whole mosquitoes. These data help inform a new generation of stable flavivirus reporter constructs to be used for high-throughput drug screens, serological diagnosis, pathogenesis studies, and transgene delivery.

### I. FLAVIVIRUS

The genus Flavivirus is a genera of the Flaviviridae family and includes the viral groups of Yellow Fever virus group, Tick-borne encephalitis virus group, Rio Bravo Group, Japanese encephalitis Group, Tyuleniy Group, Ntaya Group, Uganda S Group, Dengue Group, and Modoc Group. Members of the Flavivirus genus may produce a wide variety of disease states, such as fever, arthralgia, rash, hemorrhagic fever, and/or encephalitis. The outcome of infection is influenced by both the virus and host-specific factors, such as age, sex, genetic susceptibility, and/or pre-exposure to the same or a related agent. Some of the various diseases associated with members of the genus Flavivirus are yellow fever; dengue fever; and West Nile, Japanese, and St. Louis encephalitis. For a review of Flaviviruses see Burke and Monath (Flaviviruses. 1043-1125. In: Knipe DM., Howley PM. Field's Virology Fourth Edition Volume 1, 2001. Lippincott Williams and Wilkins, Philadelphia Pa.).

Virions of the Flaviviridae generally contain one molecule of a linear positive-sense single stranded RNA genome of approximately 10,000-11,000 nucleotides that replicates in the cytoplasm of an infected cell. Typically the 5′ end of the genome has a cap and the 3′ end that may or may not have a poly (A) tract. Some members of the genus Flavivirus are transmitted by a vector such as an insect, in many cases the insect is a mosquito.

The viral genome of the Flavivirus genus is translated as a single polyprotein and is subsequently cleaved into mature proteins. The proteins encoded by the virus typically consist of structural and non-structural proteins. Generally, there are three structural proteins that typically include the envelope protein (E protein) (amino acids 275-787 of GenBank accession number NP 041724, incorporated herein by reference), the core or capsid protein (C) (amino acids 1-92 of GenBank accession number NP 041724), and the pre-membrane protein (preM)(amino acids 105-223 of GenBank accession number NP 041724) (Yamshchikov et al., Virology 281, 294-304, 2001, incorporated herein by reference). The envelope protein is approximately 496 amino acids with an approximate molecular weight of 50 kDa and is often glycosylated. The envelope protein typically contains twelve conserved cysteine residues that form six disulfide bridges. The core protein is approximately 13 kDa and is rich in arginine and lysine residues. The pre-membrane protein is approximately 10 kDa and is cleaved during or after release of the virus from infected cells. A cleavage product of the prM protein remains associated with the virion and is approximately 8 kDa and is termed the membrane protein (M). Typically, it is the carboxy terminus of prM that remains associated with the virus particle as the M protein.

Various members of the Flaviviridae family are available through the American Type Culture Collection (Manassas Va.) under the following ATCC numbers: Dengue type 1 (VR-71), Ilheus (VR-73), Japanese encephalitis (VR-74), Murray Valley encephalitis (VR-77), Ntaya (VR-78), St. Louis encephalitis (VR-80), Uganda S (VR-81), West Nile (VR-82), Zika (VR-84), Dengue type 4 (VR-217), Dengue type 2 (VR-222), Japanese encephalitis (VR-343), Dengue type 1 (VR-344), Dengue type 2 (VR-345), Edge hill (VR-377), Entebbe bat (VR-378), Kokobera (VR-379), Stratford (VR-380), Tembusu (VR-381), Dakar bat (VR-382), Ntaya (VR-78), Banzi (VR-414), Modoc (VR-415), Rio Bravo virus (VR-416), Cowbone ridge (VR-417), Bukalasa (VR-418), Montana myotis leukoencephalitis (VR-537), Bussuquara (VR-557), Sepik (VR-906), Cowbone ridge (VR-1253), Dengue type 2 (VR-1255), Dengue type 3 (VR-1256), Dengue type 4 (VR-1257), Ilheus (VR-1258), Rio Bravo virus (VR-1263), St. Louis encephalitis (VR-1265), West Nile (VR-1267), Dengue type 4 (VR-1490), West Nile (VR-1507), and West Nile (VR-1510), each of which is incorporated herein by reference.

Examples of flaviviruses include, but are not limited to, Dengue, Japanese Encephalitis virus, West Nile Virus, Yellow Fever Virus, Tick-borne Encephalitis Virus, and the like. Additional examples of flaviviruses include, but are not limited to, Gadgets Gully virus (GGYV), Kadam virus (KADV), Kyasanur Forest disease virus (KFDV), Langat virus (LGTV), Omsk hemorrhagic fever virus (OHFV), Powassan virus (POWV), Royal Farm virus (RFV), Tick-borne encephalitis virus (TBEV), Louping ill virus (LIV), Meaban virus (MEAV), Saumarez Reef virus (SREV), Tyuleniy virus (TYUV), Aroa virus (AROAV), Dengue virus (DENV), Kedougou virus (KEDV), Japanese encephalitis virus group, Cacipacore virus (CPCV), Koutango virus (KOUV), Japanese encephalitis virus (JEV), Murray Valley encephalitis virus (MVEV), St. Louis encephalitis virus (SLEV), Usutu virus (USUV), West Nile virus (WNV), Kunjin virus, Yaounde virus (YAOV), Kokobera virus (KOKV), Ntaya virus group, Bagaza virus (BAGV), Ilheus virus (ILHV), Israel turkey meningoencephalomyelitis virus (ITV), Ntaya virus (NTAV), Tembusu virus (TMUV), Zika virus (ZIKV), Banzi virus (BANV), Bouboui virus (BOUV), Edge Hill virus (EHV), Jugra virus (JUGV), Saboya virus (SABV), Sepik virus (SEPV), Uganda S virus (UGSV), Wesselsbron virus (WESSV), Yellow fever virus (YFV), Entebbe bat virus (ENTV), Yokose virus (YOKV), Apoi virus (APOIV), Cowbone Ridge virus (CRV), Jutiapa virus (JUTV), Modoc virus (MODV), Sal Vieja virus (SVV), San Perlita virus (SPV), Rio Bravo virus group, Bukalasa bat virus (BBV), Carey Island virus (CIV), Dakar bat virus (DBV), Montana myotis leukoencephalitis virus (MMLV), Phnom Penh bat virus (PPBV), and Rio Bravo virus (RBV). Many flavivirus can also include different serotypes. For example, Dengue virus has at least four known serotypes, which include, but are not limited to, DENV1, DENV2, DENV3, and DENV4.

The flavivirus nucleic acid sequences described herein can also be included in genetic constructs, nucleic acid constructs, or expression constructs. As used herein, the terms “genetic construct,” “nucleic acid construct,” and “expression construct” are used interchangeably and refer to the DNA or RNA molecules that comprise a nucleotide sequence which encodes one or more proteins. The coding sequence, or “encoding nucleic acid sequence,” includes initiation and termination signals operably linked to regulatory elements including a promoter and polyadenylation signal capable of directing expression in a host cell or a cell of an individual to whom the nucleic acid molecule is administered.

As used herein, the term “expressible form” refers to nucleic acid constructs that contain the necessary regulatory elements operably linked to a coding sequence that encodes a protein such that when present in a host cell, the coding sequence will be expressed.

In some embodiments, the present invention provides a nucleic acid sequence encoding a replicon of a flavivirus. In some embodiments, a nucleic acid sequence encoding a replicon of the flavivirus comprises a flavivirus genome capable of self-replication. The nucleic acid molecule can be either DNA or RNA. In some embodiments, the nucleic acid sequence is free of RNA bases. In some embodiments, the DNA encoding the replicon is a plasmid. In some embodiments, the DNA is free of a virus or is not packaged in a virus. In other embodiments the nucleic acid is package in virus particle.

In certain aspects, any replicon can be used. A nucleic acid molecule that codes for all the proteins necessary for its replication in a cell is termed a “replicon”. Various types of replicons can be used including, but not limited to, a plasmid carrying a DNA-based version of a replicon that could be transfected into a cell directly (rather than an RNA transcript from the DNA) has been described for West Nile virus (Pierson, et al. (2005), Virology). Replication-competent clones of West Nile virus have also been described that carry a green fluorescent protein (GFP) reporter virus (Pierson, et al. (2005), Virology, 334:28-40). In some embodiments, the DENV replicon is the replicon described and used in Ansarah-Sobrinho et al. (2008), Virology 381, 67-74. In some embodiments, the replicon is a DENV2 replicon. In some embodiments, the replicon is a DENV1, DENV3, or a DENV4 replicon.

The nucleic acid sequences described herein and throughout can comprise a promoter operably linked to the nucleic acid sequence encoding one or more proteins. The proteins can be, but not limited to, C, prME and/or the replicon of the flavivirus. The promoter can be any promoter, including but not limited to promoters that are functional in eukaryotic cells. In some embodiments, the promoter is specifically functional in a eukaryotic cell. In some embodiments, the promoter is, but not limited to a CMV promoter, SV40, and the like. In some embodiments, the promoter is an inducible promoter. In other embodiments the promoter(s) are flavivirus promoter(s).

The nucleic acid sequence encoding replicons and the resulting replicons of the present invention can also comprise a reporter such that one can monitor the replication or expression of the genes found in the nucleic acid sequence of the replicon. The reporter can also be used to measure infectivity of any virus or virus-like particle that contains the replicon. Examples of reporters include, but are not limited to, a fluorescent reporter or an enzymatic reporter. Examples of enzymatic reporters include, but are not limited to, a luciferase reporter, (3-Galactosidase reporter, alkaline phosphatase reporter, chloramphenicol acetyltransferase (CAT), and the like. Examples of fluorescent reporters include, but are not limited to GFP (green fluorescent protein) reporter, RFP (red fluorescent protein) reporter, YFP (yellow fluorescent protein) reporter, nsTGP, and the like. Examples of luciferase reporters include, but are not limited to nano-Luc reporter, renilla luciferase reporter and firefly luciferase reporter. In some embodiments the replicon comprises a gene that allows for selection of a cell that comprises the replicon. In certain embodiment a reporter polypeptide comprises, consists essentially of, or consists of an amino acid sequence of SEQ ID NO:2, 4, 6, 8, 10, or 12. In certain aspects, the reporter and can be a protein having 95, 96, 97, 98, 99, or 100% identity to SEQ ID NO:2, 4, 6, 8, 10, or 12. In other aspects, the reporter can comprise 1, 2, 3, 4, 5, 6, 7, 8, 9, 10, 11, 12, 13, 14, 15, 16, 17, 18, 19, or 20 amino acid substitutions to the amino acid sequence of SEQ ID NO:2, 4, 6, 8, 10, or 12 as long as he operability of the protein as a reporter is not compromised. For example, a cell can be selected for comprising the nucleic acid sequence encoding the replicon by contacting the cell with a drug or chemical that because of the presence of the replicon the cell is resistant to the drug or chemical whereas cells that do not contain the replicon will die. Accordingly, in some embodiments, the nucleic acid sequence encoding the replicon comprises a drug resistant gene that allows a cell to escape the effects of drug or chemical. Examples of markers that can be used include, but are not limited to, zeomycin, and the like. Zeocin (zeomycin) is a member of the bleomycin antibiotic family. One could also use hygromycin, neomycin, blasticidin, puromycin, or mycophenolic acid resistance markers and antibiotics and the like as selection markers.

The present invention also provides methods of producing flavivirus reporter virus particles (RVPs). A reporter virus particle is a particle that comprises elements of a virus which are produced from a cell comprising a replicon and comprising any other elements necessary for the generation of the virus or virus-like particle. The RVP also comprises a reporter gene. The presence of the reporter gene can be used to monitor the particle's assembly, replication, infection ability, and the like.

In some embodiments, a method of producing flavivirus RVPs comprises contacting a cell with a nucleic acid sequence encoding a replicon. In some embodiments, the nucleic acid molecule encoding a replicon comprises a DNA molecule that encodes an RNA sequence. The RVPs are then produced once the cell has taken up the replicon. The nucleic acid sequence can be any sequence described herein.

The nucleic acid molecule encoding the replicon can be contacted with the cell in any manner that enables the nucleic acid molecule encoding the replicon to enter the cell or to be transfected into the cell. Examples of methods of contacting a nucleic acid molecule encoding the replicon with a cell includes, but are not limited to, calcium phosphate transfection, lipid-mediated transfection, PEI-mediated transfection, electroporation, infection with a virus coding for the replicon, and the like.

In some embodiments, the cell that is contacted with the nucleic acid encoding a replicon comprises elements that can express the structural elements of the flavivirus such that when the replicon is expressed in the cell in conjunction with the structural elements, a RVP is produced. In some embodiments, the structural elements are stably expressed in the cell. Examples of structural elements that can be present in the producer cell include, but are not limited to, Capsid (C), pre-membrane protein (prM), Envelope protein (E), or combinations thereof.

In some embodiments the structural proteins are under control of an inducible promoter such that the expression is regulated by the presence or absence of a compound or other type of molecule. Any inducible promoter can be used. Examples of inducible promoters include, but are not limited to, tetracycline (TREx, Invitrogen), Rheoswitch (NEB), Ecdyson (Invitrogen, Stratagene), Cumate (Qbiogene), glucocorticoid responsive promoter, and the like.

The present invention also provides compositions comprising flavivirus reporter virus particles and a storage buffer. The storage buffer is any buffer that allows the flavivirus reporter virus particles to be stored (e.g. frozen or refrigerated) for a period of time and the falvivirus reporter virus particles maintain their ability to infect a flavivirus susceptible cell (e.g. a cell that can be infected by a flavivirus virus or RVP). In some embodiments, the storage buffer is maintained at a pH of about 7.5 to about 8.5.

The present invention also provides a method of identifying a compound that can inhibit flavivirus infection. In some embodiments, the method comprises contacting a cell with a flavivirus described herein in the presence or absence of a test compound and determining if the flavivirus infects the cell in the presence and absence of a test compound. If the flavivirus infects the cell in the absence of the test compound, but not in the presence of the test compound, the test compound is said to be a compound that inhibits or modulates infection. The test compound that can inhibit flavivirus infection can be any type of compound or molecule including, but not limited to, a small organic molecule, small peptides, fusions of organic molecules and peptides, and the like.

The present invention also provides methods of identifying a compound that inhibits flavivirus assembly comprising contacting a flavivirus producer cell with a test compound and determining if the flavivirus can assemble in the presence of said test compound. A compound that inhibits assembly can be any compound including but not necessarily limited to small organic compounds, peptides, complete antibodies, any portion of antibody, or fusion compounds of any combination thereof

As used herein “codon optimized,” refers to a nucleic acid sequence that has been modified where the one or more modification results in increased protein expression as compared to the unmodified nucleic acid sequence. Codon optimization also refers to where a nucleic acid sequence is modified that results in enhanced expression in a eukaryotic cell by replacing one or more codons of the native sequence with codons that are more frequently or most frequently used in the genes of the eukaryotic cell. Various species exhibit particular bias for certain codons of a particular amino acid. In some embodiments, the increase in expression (e.g. protein expression) is at least 10%, 20%, 30%, 40%, 50%, 60%, 70%, 80%, 90% more than the non-optimized sequence. In some embodiments, the increase in expression is at least 100%, 200%, 300%, 400% or 500% more than the non-optimized sequence. The comparison can be made using any technique. The unmodified nucleic acid sequence can also be referred to as the wild-type sequence or a sequence that is found in nature.

Most amino acids are encoded by multiple synonymous codons. Different organisms use synonymous codons with different frequencies, a phenomenon known as codon bias. Preferred codon usage has been identified as an important factor in the efficiency of both prokaryotic and eukaryotic gene expression (Gustafsson et al., Trends in Biotechnology 22(7), 2004). The presence of infrequently used codons in messenger RNAs can result in decreased expression of exogenous genes. This is especially true for the expression of viral genes, which are often A-T rich and encode cis-acting negative regulatory sequences (Gustafsson et al., Trends in Biotechnology 22(7), 2004).

Codon optimization is a methodology used to improve heterologous protein expression by altering the codon usage of target genes. Codon optimization introduces silent nucleotide changes to remove rare codons and replace them with more commonly used codons that reflect the codon bias of a given host cell. Additionally, codon optimization employs computer algorithms to analyze gene sequences for silent nucleotide changes that can improve a variety of important factors involved in different stages of protein expression including mRNA secondary structure, mRNA nuclear export, and translational cis-elements. Codon optimization of several viral genes, including the E5 gene of human papillomavirus (HPV) and the U51 gene from human herpesvirus (HHV), results in enhanced protein expression in mammalian cells (Disbrow et al., Virology 311, 2003; Bradel-Tretheway et al., Journal of Virological Methods 111, 2003).

The present invention also provides host cells and isolated cells comprising any nucleic acid molecule described herein.

In some embodiments, the present invention provides flavivirus or flavivirus particles comprising the nucleic acid molecules described herein.

### II. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. EXAMPLE 1 - Identifying Optimal Capsid Duplication Length for the Stability of Reporter Flaviviruses

1. Materials and methods

Viruses and cells. Zika virus strain Dakar 41525, yellow fever 17D strain YFS11, dengue virus 1 strain Western Pacific, dengue virus 2 strain New Guinea C, Japanese encephalitis virus 14-14-2 strain 1454, and West Nile virus strain NY99 with NS5 E218A 25,26 were cloned into full-length plasmids using the low copy pCC1 vector as has been previously described²⁷. NEBuilder HiFi DNA Assembly mix (NEB E2621) was used to assemble all plasmids. Full length dengue virus 3 strain VN32 and dengue virus 4 strain MY01 transcripts were assembled from four and three fragments, respectively, by in vitro ligation. Viruses were recovered after electroporation (Biorad GenePulser Xcell) of in vitro transcribed RNAs in Vero (ATCC Cat# CCL-81) or BHK21 (ATCC Cat# CCL-10) cells as previously described²⁰. Vero and BHK21 cells were grown in Dulbecco's Modified Eagle Medium (Gibco 11965) with 10% fetal bovine serum (FBS, Hyclone SH30071) and 1% penicillin/streptomycin (Gibco 15140). Huh7 cells were grown in Dulbecco's Modified Eagle Medium with Glutamax (Gibco 10566) supplemented with 10% FBS, 1% penicillin/streptomycin, and 1% non-essential amino acids (Gibco 11140). Infections were carried out in the same media excepting supplementation with 2% fetal bovine serum instead of 10%. Vero, BHK21, and Huh7 cells were grown at 37° C. in a humidified incubator with 5% CO2.

Immunofluorescence assay. Vero cells were seeded into chamber slides immediately post-electroporation. At the indicated time points, cells were washed with PBS and fixed with cold methanol at −30° C. for >30 minutes. Slides were washed with PBS and blocked with PBS+1% FBS overnight at 4° C. The flavivirus envelope reactive antibody 4G2 (ATCC Cat# HB-112), diluted in blocking buffer, was used as a primary antibody incubated for 1 hour at room temperature. A secondary goat anti-mouse IgG antibody conjugated with Alexa Fluor 488 (Thermo Fisher Scientific Cat# A-11001) was then used to probe for 4G2 for 1 hour. Slides were then washed 3× with PBS and stained with DAPI (Vector Laboratories, H-1200) and then imaged on a Nikon Eclipse Ti2 microscope. ImageJ (NIH) was used to process the images.

Focus forming assay. All viruses were titered by focus-forming assay. Viruses were serially diluted ten-fold in DMEM supplemented with 2% FBS and used to infect Vero cells that had been seeded the day previously at 2×10⁵ cells per well in a 24-well plate. After a 1-h infection with rocking every 15 minutes, the virus was removed and methylcellulose/DMEM overlay was added. After four days of infection, the overlay was removed, and cells were fixed with a 1:1 solution of methanol/acetone for >15 minutes. Plates were then washed 3× with PBS, blocked with PBS+3% FBS for 30 minutes, and then incubated with virus-specific mouse immune ascites fluid (MIAF, World Reference Center for Emerging Viruses and Arboviruses, UTMB). After >1-h incubation with MIAF, plates were washed with PBS and incubated with a horseradish peroxidase-conjugated anti-mouse IgG antibody (SeraCare KPL Cat#474-1806). After a 3X PBS wash, foci were developed in the dark using an AEC peroxidase substrate kit (Enzo 43825) according to the manufacturer's protocol and imaged using a BioRad ChemiDoc Imaging System.

Reporter virus passaging and stability. Virus recovered from electroporation was termed P0. 500 μL of this was added to a T75 flask with a confluent layer of Vero cells. The infection was discontinued once cell death was observed after which media was harvested. 500 of the new passage was then used to infect a new T75 flask for the next passage. This was carried out in parallel series for each virus. Stability was assessed by isolating viral RNA (Qiagen 52904 or TriZOL, Invitrogen 15596026) from each passage and using this as a template for an RT-PCR reaction (Invitrogen 12574) with primers that spanned the 5′ UTR to the end of the capsid. The products were then run on a 0.6% agarose gel and imaged with a BioRad GelDoc EZ Imager.

Growth kinetics by focus forming assay. Six well plates were seeded at 8×10⁵ cells per well with Vero cells the day before infection. Cells were infected at a MOI of 0.01 in triplicate for 1 h with shaking every 15 minutes, followed by a 3X PBS wash and addition of media supplemented with 2% FBS. Supernatant samples were taken at 24, 48, 72, 96, and 120 h and titered by focus-forming assay.

Reporter neutralization assays. Reporter neutralization tests were done by two-fold serially diluting sera, starting at 1:50 in DMEM with 2% FBS. Sera samples positive for ZIKV and DENV1-4 were pooled from mice infected with the respective virus. YFV and JEV sera samples were from vaccinated mice. Sera dilutions were mixed 1:1 by volume with the respective reporter virus and incubated at 37° C. for 1 h. The virus/sera mixture was then plated on Vero cells in a white, opaque 96 well plate that was seeded at 1.5×10⁴ cells per well. After a 4-h incubation at 37° C., the wells were washed 2× with PBS and 50 μL of NanoGlo substrate diluted 1:50 in NanoGlo Assay Buffer was added to the cells. Plates were read in a BioTek Cytation 5 plate reader after 3 minutes with a gain of 150. Positive controls consisted of virus infection with no sera. Negative controls comprised virus plated in wells with no cells. This negative control allows for subtraction of background luciferase signals from the virus media. After subtraction of negative controls, values were converted to a percent of the positive control. The data was then analyzed by four parameter nonlinear regression, with the top and bottom constrained to 100 and zero, respectively, and the NT50 reported.

Antiviral assays. The panflavivirus inhibitor NITD008 was diluted in 90% DMSO to 10 μM and then two-fold serially diluted 8 times. These dilutions were mixed with virus (MOI 0.1-DENV1, DENV2, DENV3; MOI 0.01-ZIKV; MOI 0.001-DENV4, YFV, JEV) and plated on Huh? cells that were seeded at 1.5×10⁴ cells per well the previous day in media with 2% FBS. Cells were washed 48 h post infection three times with PBS followed by addition of NanoGlo substrate diluted 1:100 in NanoGlo Assay Buffer. Plates were read by a BioTek Cytation 5 plate reader after 3 minutes.

Mosquito infections. For the micro-injection study, Rockefeller strain Aedes aegypti mosquitoes were injected (100 nL) intrathoracically with virus diluted in PBS so that each mosquito received 50 FFU. Mosquitoes were cultured at 28° C. for 8, 12, and 18 days. For blood feeding study, sheep's blood was centrifuged at 1000g, 4° C. for 20 minutes to separate plasma and cells. The plasma was heat-inactivated at 56° C. for 1 hour and the cells were washed twice with PBS after which they were combined again. The blood was spiked with virus to a concentration of 2×10⁶FFU/mL. Engorged mosquitoes were further reared and harvested at 8, 12, and 18 days. At the time points indicated mosquito samples from both experiments were thoroughly homogenized (Qiagen TissueLyser II) in 200₁..t.L PBS and centrifuged to pellet the tissue. 50₁..t.L of supernatant was used for both RT-qPCR and luciferase assay. RNA was harvested using Qiagen RNeasy minikit and used in a Taqman RT-qPCR reaction targeting a region in NS5. Aedes aegypti actin served as a control. For luciferase assay, 50₁..t.L of supernatant from homogenized mosquito was added to a 96 well opaque white plate, followed by addition of NanoGlo substrate, diluted 1:50 in NanoGlo Assay Buffer. Samples were read in a BioTek Cytation 5 plate reader. Background luciferase levels (no mosquitoes) and clean, uninfected mosquitoes were used as negative controls.

Statistical analysis. Graphpad Prism 8 was used for graphing and statistical analysis. Statistical tests used, as well as significance levels, are noted in the figure legends. All replicated values are shown on each graph.

2. Results

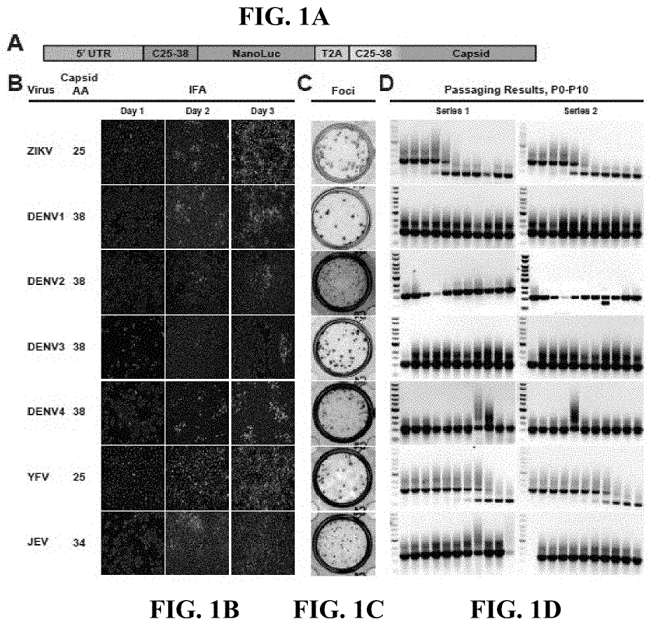

Panel of reporter flaviviruses. Reporter flaviviruses were constructed as first described by¹⁶ using capsid duplication lengths as found in the literature^{10,16,20,23,24} (FIG. 1A-B). All viruses except DENV3 and DENV4, which were assembled by in vitro ligation (FIG. 51), were constructed using traditional, plasmid-based reverse genetics approaches²⁸. DENV3 and DENV4 full genomes are difficult to clone in bacteria, due to putative toxic elements present 29′30.

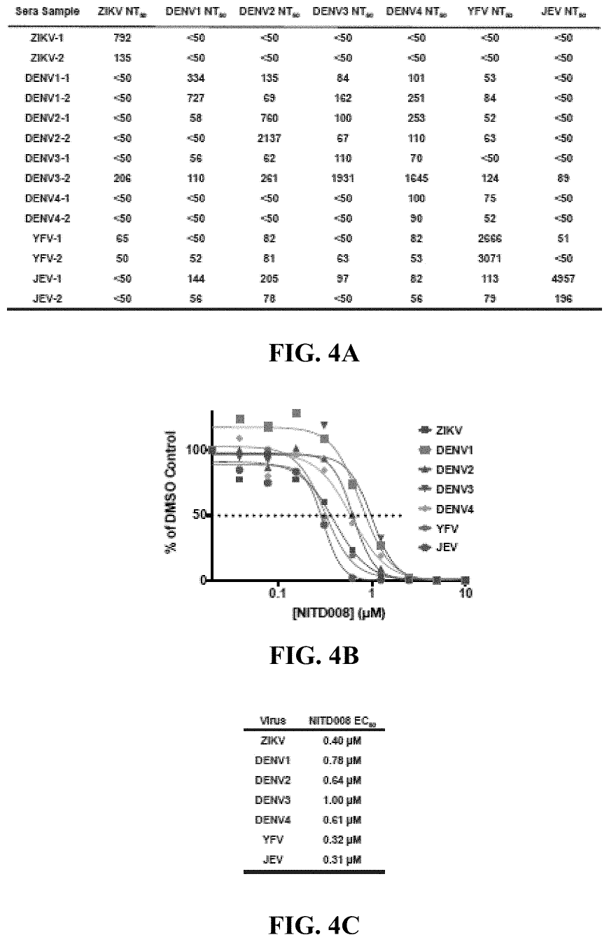

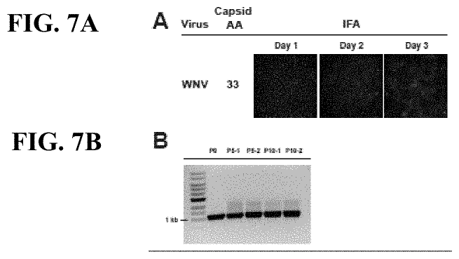

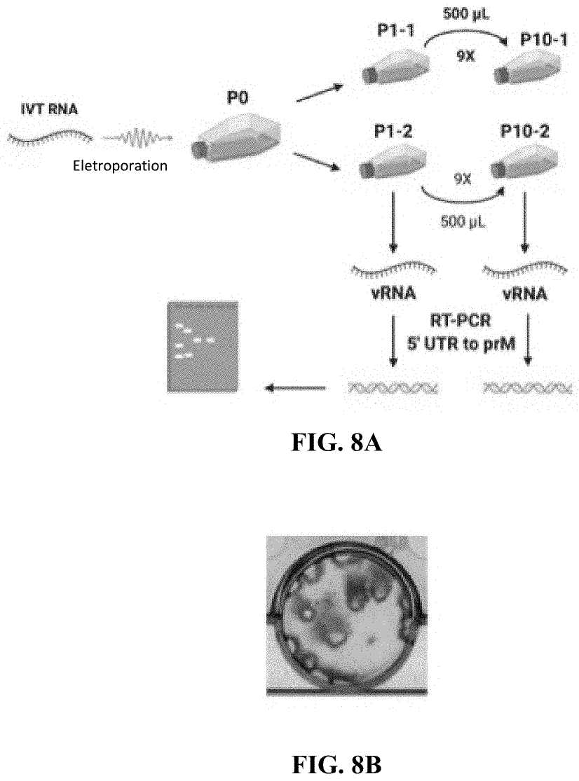

The NanoLuc gene followed by the 2A sequence from thosea asigna virus (T2A) were inserted between a duplicated portion of the capsid and the full-length capsid. To help prevent homologous recombination, the codons in the capsid sequence corresponding to the duplicated portion were scrambled to reduce homology. All full-length DNAs were used as a template in an in vitro transcription reaction to generate full-length RNAs, which were subsequently electroporated into Vero cells (for ZIKV, YFV, and WNV) or baby hamster kidney (BHK) cells (for DENV1-4 and JEV). Immunofluorescence assay (IFA) was used to indicate viral spread, post electroporation (FIGS. 1B and 7A). Focus-forming assay shows all reporter viruses form distinct foci (FIG. 1C), but no plaques when stained with crystal violet (data not shown). The IFA and focus-forming results corroborate with the viral replication kinetics among different versions of reporter viruses (see later replication kinetic results in FIG. 3). To assess stability, viruses were passaged ten times on Vero cells according to the scheme in FIG. 8A. RT-PCR products corresponding to each passage show consistent band size for all viruses out to P10, including West Nile virus (WNV) which results were obtained after the initial review of this manuscript (FIG. 7B). The exception to this positive outcome is ZIKV and YFV, which have the shortest capsid duplication, 25 amino acids (FIG. 1D). These two viruses showed a decrease in band size during passaging. These results suggested that the length of the capsid duplication may have an impact on virus stability.

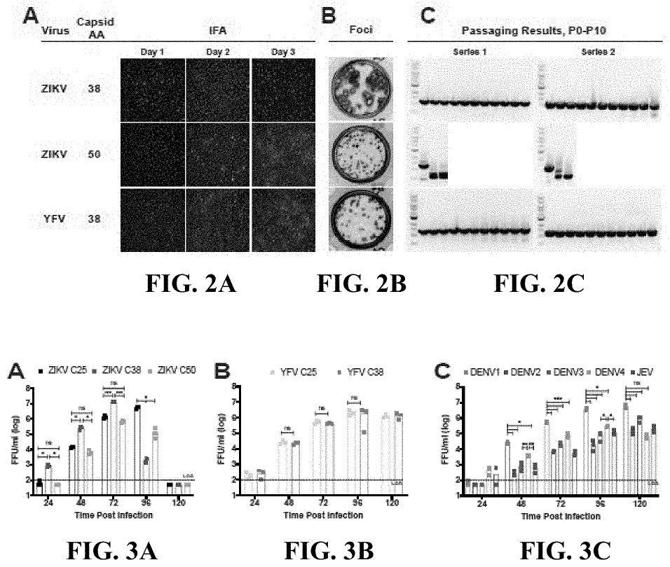

Extended capsid duplication. The results from passaging these different flaviviruses indicated that a longer capsid duplication could positively impact its stability. This hypothesis was tested by creation of ZIKV and YFV C38 NanoLuc viruses. C38 was chosen based on the robust results from DENV1-4 using this length. During this time, Volkova, et al., published a report on reporter ZIKV and the effect of capsid duplication size on replication. Their conclusion was that C50 is the optimal length for viral growth, with this being the shortest length that was not statistically attenuated compared to WT virus¹⁸. Based on this report, we also constructed a C50 ZIKV. IFA results post-electroporation suggested that ZIKV C38 virus replicated more robustly than the C25 and C50 virus, while YFV C38 showed little difference compared to YFV C25 (FIG. 2A, compare to FIG. 1B). Focus size (FIG. 2B) comparison between ZIKV C25 and ZIKV C50 showed little difference but, ZIKV C38 formed clear, larger plaques similar to non-reporter wild-type ZIKV (FIG. 8B). YFV C38 focus size was only slightly larger than YFV C25. The C38 ZIKV and YFV and C50 ZIKV were continuously passaged and analyzed for reporter gene stability by RT-PCR (FIG. 2C). Unexpectedly, ZIKV C50 showed early instability, so only passaging results from PO-P2 are shown. In contrast, ZIKV and YFV C38 were stable after ten rounds of continuous cell culture. These results suggest that an optimal length of duplicated capsid sequence (e.g., C38) is required for reporter virus stability. Under such condition, the frameshift or other mutations in the duplicated capsid region is not required for the stability of reporter virus.

Effect of extended capsid duplication on viral growth. The effect of capsid duplication length on viral growth was assessed on Vero cells. Cells were infected with ZIKV C25, C38, and C50 at a MOI of 0.01 and assessed at 24, 48, 72, 96, and 120 h post infection by focus forming assay (FIG. 3A). ZIKV C38 replicated to significantly higher titers than ZIKV C25 and ZIKV C50, reaching 10⁷ FFU/mL, at 24, 48, and 72 h post infection. ZIKV C25 and ZIKV C50 growth are similar across the same time period, though ZIKV C25 titers did continue to increase until 96 h. Conversely, growth comparison of YFV C25 and YFV C38 showed no significant difference at any time point, despite YFV C38's increased stability (FIG. 3B). DENV1-4 and JEV growth kinetics on Vero cells (MOI 0.01) show that DENV1 replicated significantly higher at times 24-96 h post infection (FIG. 3C). Together these data show that among C25, C38, and C50, C38 is the most optimal capsid duplication length for ZIKV replication. In contrast to these results, there seems to be no replication advantage for YFV C38 over YFV C25.

To directly examine the effect of reporter gene insertion on viral replication, we compared the replication kinetics between the parental wild-type ZIKV and the reporter ZIKV C38 on Vero cells (FIG. 8). The results showed that replication between the two is similar at all time points but 72 h, where WT virus is 10-fold lower, possibly due to death of the host cells.

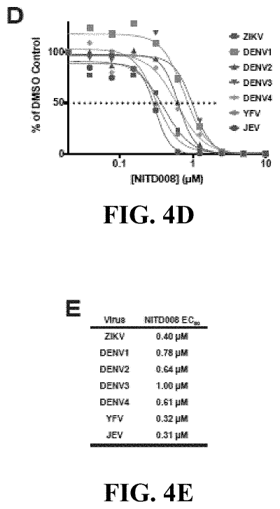

Rapid neutralization tests and antiviral discovery. One of the aims of this study was to develop stable reporter flaviviruses for neutralization tests and antiviral compound assays. As we have previously done, the stable reporter viruses were used to test a panel of flavivirus-immune mouse sera in a four-hour neutralization test. Reporter virus neutralization scheme. Vero cells were seeded in an opaque, white 96 well plate at 1.5×10⁴ cells per well the day before. Sera samples were two-fold serially diluted, starting at 1:50 and then mixed with equal volume of virus and incubated for 1 h at 37° C. The sera:virus mixture was then used to inoculate Vero cells in a 96 well plate. After 4 h incubation at 37° C., plates were washed twice with PBS and then NanoGlo substrate was added and luciferase levels read by plate reader. NT50 results are indicative of the previous infection with the homologous virus yielding the highest NT50, though some cross-neutralization by heterologous viruses was observed (FIG. 4A). Using the flavivirus inhibitor NITD008^{31,32}, each virus was used in an antiviral compound assay. Antiviral assay scheme. Huh7 cells (1.5×10⁴ cells per well) were seeded the previous day. NITD008 was two-fold serially diluted starting at 10 μM. The dilutions were mixed with virus and plated on the Huh7 cells in a white, opaque 96 well plate and incubated at 37° C. for 48 h. Following a 3X PBS wash, results were read by plate reader after addition of NanoGlo substrate. Increasing concentrations of NITD008 decreased luciferase expression compared to control (FIG. 4B) resulting in potent EC50 values (FIG. 4C). These results support our previous data showing that NanoLuc-tagged flaviviruses can be valuable tools in rapid sero-diagnostic assays and antiviral compound screens.

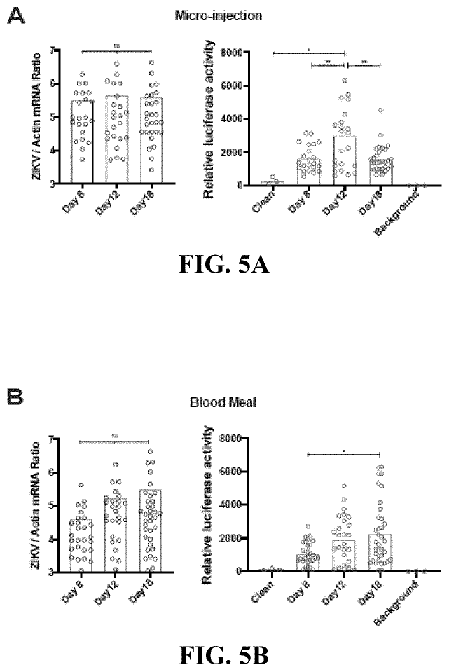

ZIKV C38 in mosquitoes. Reporter virus use in mosquito experiments is highly advantageous, where RNA extraction from individual mosquitoes is time-consuming. Intrathoracic injection of mosquitoes with ZIKV C25 showed no viral replication and no luciferase expression (data not shown), possibly due to reporter gene induced replication attenuation. Because ZIKV C38 had more robust luciferase expression compared to C25 in C6/36 cells (data not shown), we characterized ZIKV C38 replication in whole Aedes aegypti mosquitoes by both micro-injection, which surpasses the midgut barrier, and membrane blood feeding. Mosquitoes were micro-injected in the thorax with 50 FFU ZIKV C38 and at days 8, 12, and 18, whole mosquitoes were homogenized in PBS and assayed by both qPCR (FIG. 5A, left panel) and luciferase assay (right panel). Although viral RNA did not increase from day 8 to day 12, the luciferase assay shows a statistically significant peak at day 12. In a separate experiment, mosquitoes were allowed to feed on blood spiked with 2×10⁶ FFU/mL ZIKV C38. These mosquitoes were also homogenized on days 8, 12, and 18 and evaluated by both qPCR and luciferase assay (FIG. 4B, right and left panel, respectively). By blood feeding, ZIKV C38 titers increased at each time point by qPCR, though the increase was not statistically significant. Corroboratively, the luciferase activities significantly increased from day 8 to 18. Uninfected mosquitoes were also assayed in FIG. 5B (right panel) as a negative control. These data show that ZIKV C38 replicates in Aedes aegypti mosquitoes and luciferase output can be used to assay viral replication.

### REFERENCES

1. Chong HY, Leow CY, Maj eed ABA, Leow CH. Flavivirus infection-A review of immunopathogenesis, immunological response, and immunodiagnosis. Virus Res 2019; 274: 197770.
2. Bhatt S, Gething PW, Brady OJ et al. The global distribution and burden of dengue. Nature 2013; 496: 504.
3. Mathers CD, Ezzati M, Lopez AD. Measuring the Burden of Neglected Tropical Diseases:

The Global Burden of Disease Framework. Plos Neglect Trop D 2007; 1: e114.
4. Schuler-Faccini L, Ribeiro EM, Feitosa IML et al. Possible Association Between Zika Virus

Infection and Microcephaly - Brazil, 2015. Mmwr Morbidity Mortal Wkly Rep 2016; 65: 59-62.
5. Barrett ADT, Higgs S. Yellow Fever: A Disease that Has Yet to be Conquered. Annu Rev

Entomol 2007; 52: 209-229.
6. Ishikawa T, Yamanaka A, Konishi E. A review of successful flavivirus vaccines and the problems with those flaviviruses for which vaccines are not yet available. Vaccine 2014; 32: 1326-1337.
7. Guy B, Noriega F, Ochiai RL et al. A recombinant live attenuated tetravalent vaccine for the prevention of dengue. Expert Rev Vaccines 2017; 16: 1-13.
8. Yun S-I, Kim S-Y, Rice CM, Lee Y-M. Development and Application of a Reverse Genetics

System for Japanese Encephalitis Virus. J Virol 2003; 77: 6450-6465.
9. Puig-Basagoiti F, Deas TS, Ren P, Tilgner M, Ferguson DM, Shi P-Y. High-Throughput

Assays Using a Luciferase-Expressing Replicon, Virus-Like Particles, and Full-Length Virus for West Nile Virus Drug Discovery. Antimicrob Agents Ch 2005; 49: 4980-4988.
10. Zou G, Xu HY, Qing M, Wang Q-Y, Shi P-Y. Development and characterization of a stable luciferase dengue virus for high-throughput screening. Antivir Res 2011; 91: 11-19.
11. Schoggins JW, Wilson SJ, Panis M et al. A diverse range of gene products are effectors of the type I interferon antiviral response. Nature 2011; 472: 481-485.
12. Pierson TC, Sanchez MD, Puffer BA et al. A rapid and quantitative assay for measuring antibody-mediated neutralization of West Nile virus infection. Virology 2006; 346: 53-65.
13. Shan C, Xie X, Ren P et al. A Rapid Zika Diagnostic Assay to Measure Neutralizing

Antibodies in Patients. Ebiomedicine 2017; 17: 157-162.
14. Pierson TC, Diamond MS, Ahmed AA et al. An infectious West Nile Virus that expresses a

GFP reporter gene. Virology 2005; 334: 28-40.
15. Samsa MM, Mondotte JA, Caramelo JJ, Gamarnik AV. Uncoupling cis-Acting RNA

Elements from Coding Sequences Revealed a Requirement of the N-Terminal Region of Dengue Virus Capsid Protein in Virus Particle Formation. J Virol 2012; 86: 1046-1058.
16. Shustov AV, Mason PW, Frolov I. Production of Pseudoinfectious Yellow Fever Virus with a Two-Component Genome v. J Virol 2007; 81: 11737-11748.
17. Mutso M, Saul S, Rausalu K et al. Reverse genetic system, genetically stable reporter viruses and packaged subgenomic replicon based on a Brazilian Zika virus isolate. J Gen Virol 2017; 98: 2712-2724.
18. Volkova E, Tsetsarkin KA, Sippert E et al. Novel Approach for Insertion of Heterologous

Sequences into Full-Length ZIKV Genome Results in Superior Level of Gene Expression and Insert Stability. Viruses 2020; 12: 61.
19. Baker C, Xie X, Zou J, Muruato A, Fink K, Shi P-Y. Using recombination-dependent lethal mutations to stabilize reporter flaviviruses for rapid serodiagnosis and drug discovery. Ebiomedicine 2020; 57: 102838.
20. Shan C, Xie X, Muruato AE et al. An Infectious cDNA Clone of Zika Virus to Study Viral

Virulence, Mosquito Transmission, and Antiviral Inhibitors. Cell Host Microbe 2016; 19: 891-900.
21. Schoggins JW, Dorner M, Feulner M et al. Dengue reporter viruses reveal viral dynamics in interferon receptor-deficient mice and sensitivity to interferon effectors in vitro. P Natl Acad Sci Usa 2012; 109: 14610-5.
22. Samsa MM, Mondotte JA, Iglesias NG et al. Dengue virus capsid protein usurps lipid droplets for viral particle formation. Plos Pathog 2009; 5: e1000632.
23. Zhang P-T, Shan C, Li X-D et al. Generation of a recombinant West Nile virus stably expressing the Gaussia luciferase for neutralization assay. Virus Res 2016; 211: 17-24.
24. Li X-F, Li X-D, Deng C-L et al. Visualization of a neurotropic flavivirus infection in mouse reveals unique viscerotropism controlled by host type I interferon signaling. Theranostics 2017; 7: 912-925.
25. Daffis S, Szretter KJ, Schriewer J et al. 2′-O methylation of the viral mRNA cap evades host restriction by IFIT family members. Nature 2010; 468: 452-6.
26. Kaiser JA, Luo H, Widen SG et al. Genotypic and phenotypic characterization of West Nile virus NS5 methyltransferase mutants. Vaccine 2019; 37: 7155-7164.
27. Zou J, Xie X, Luo H et al. A single-dose plasmid-launched live-attenuated Zika vaccine induces protective immunity. Ebiomedicine 2018; 36: 92-102.
28. Shi P-Y, Tilgner M, Lo MK, Kent KA, Bernard KA. Infectious cDNA Clone of the Epidemic

West Nile Virus from New York City. J Virol 2002; 76: 5847-5856.
29. Gallichotte EN, Widman DG, Yount BL et al. A new quaternary structure epitope on dengue virus serotype 2 is the target of durable type-specific neutralizing antibodies. Mbio 2015; 6: e01461-15.
30. Messer WB, Yount B, Hacker KE et al. Development and characterization of a reverse genetic system for studying dengue virus serotype 3 strain variation and neutralization. Plos Neglect Trop D 2012; 6: e1486.
31. Yin Z, Chen Y-L, Schul W et al. An adenosine nucleoside inhibitor of dengue virus. P Natl

Acad Sci Usa 2009; 106: 20435-9.
32. Deng Y-Q, Zhang N-N, Li C-F et al. Adenosine Analog NITD008 Is a Potent Inhibitor of

Zika Virus. Open Forum Infect Dis 2016; 3: ofw175.
33. Deas TS, Binduga-Gajewska I, Tilgner M et al. Inhibition of Flavivirus Infections by

Antisense Oligomers Specifically Suppressing Viral Translation and RNA Replication. J Virol 2005; 79: 4599-4609.
34. Xie X, Muruato AE, Zhang X et al. A nanoluciferase SARS-CoV-2 for rapid neutralization testing and screening of anti-infective drugs for COVID-19. Nat Commun 2020; Submitted.
35. Muruato AE, Fontes-Garfias CR, Ren P et al. A high-throughput neutralizing antibody assay for COVID-19 diagnosis and vaccine evaluation. Nat Commun 2020; In press.
36. Filomatori CV, Lodeiro MF, Alvarez DE, Samsa MM, Pietrasanta L, Gamarnik AV. A 5′

RNA element promotes dengue virus RNA synthesis on a circular genome. Gene Dev 2006; 20: 2238-2249.
37. Clyde K, Harris E. RNA Secondary Structure in the Coding Region of Dengue Virus Type 2

Directs Translation Start Codon Selection and Is Required for Viral Replication. J Virol 2006; 80: 2170-2182.
38. Friebe P, Harris E. Interplay of RNA Elements in the Dengue Virus 5′ and 3′ Ends Required for Viral RNA Replication v. J Virol 2010; 84: 6103-6118.
39. Liu Z-Y, Li X-F, Jiang T et al. Novel cis-acting element within the capsid-coding region enhances flavivirus viral-RNA replication by regulating genome cyclization. J Virol 2013; 87: 6804-18.
40. Li P, Wei Y, Mei M et al. Integrative Analysis of Zika Virus Genome RNA Structure Reveals Critical Determinants of Viral Infectivity. Cell Host Microbe 2018; 24: 875-886.e5.
41. Borba L de, Villordo SM, Iglesias NG, Filomatori CV, Gebhard LG, Gamarnik AV.

Overlapping Local and Long-Range RNA-RNA Interactions Modulate Dengue Virus Genome Cyclization and Replication. J Virol 2015; 89: 3430-3437.
42. Shang Z, Song H, Shi Y, Qi J, Gao GF. Crystal Structure of the Capsid Protein from Zika Virus. J Mot Blot 2018; 430: 948-962.

1. A recombinant flavivirus comprising:
a heterologous reporter cassette, the heterologous reporter cassette having a 5′ end, a nucleotide segment encoding a reporter, and a 3′ end;
the 5′ end of the reporter cassette encoding 25 to 38 amino acids of an amino terminus of flavivirus capsid protein;
the 3′ end of the reporter cassette encoding 25 to 38 amino acids of an amino terminus of the flavivirus capsid protein, wherein the recombinant flavivirus is stable.

2. The recombinant flavivirus of claim 1, further comprising a nucleic acid segment encoding a 2A self-cleaving sequence position immediately 3′ of the reporter and immediately 5′ of the nucleotide segment encoding the second 25 to 38 amino acids of a flavivirus capsid protein.

3. The recombinant flavivirus of claim 2, wherein the 2A self-cleaving segment is a T2A segment.

4. The recombinant flavivirus of claim 1, wherein the flavivirus is a Dengue virus, Zika virus, or Japanese encephalitis virus.

5. The recombinant flavivirus of claim 1, wherein the reporter is a NanoLuc, EGFP, mCherry, mScarlet, mNeonGreen, or Renilla Luciferase reporter.

6. The recombinant flavivirus of claim 1, wherein the flavivirus has a nucleic sequence that is at least 98% identical to SEQ ID NO:13, 15, 17, 19, 21, 23, 25, 27, 29, or 31.

7. The recombinant flavivirus of claim 1, wherein the flavivirus encodes a polyprotein that is at least 95% identical to the amino acid sequence of SEQ ID NO:14, 16, 18, 20, 22, 24, 26, 28, 30, or 32.

8. A recombinant flavivirus polyprotein comprising an amino terminal segment comprising a heterologous reporter, the reporter being flanked by an amino terminal first capsid segment corresponding to capsid segment comprising 25 to 38 amino terminal amino acids of the flavivirus capsid protein and a carboxy terminal second capsid segment corresponding to a capsid segment comprising 25 to 38 amino of the amino terminus of the flavivirus capsid protein.

9. The polyprotein of claim 8, further comprising a 2A self-cleaving segment between the reporter and the second capsid segment.
